(19) United States
(12) Patent Application Publication
(10) Pub. No.: US 2004/0127909 A1
(43) Pub. Date: Jul. 1, 2004

(54) Threaded dental or medical implants

(76) Inventor: Vincent J. Morgan, Boston, MA (US)

Correspondence Address: John A. Haug, PO Box 386, West Harwich, MA, 02671, US

(21) Appl. No.: 10/736,051

(22) Filed: Dec. 15, 2003

Related U.S. Application Data
(60) Provisional application No. 60/433,659, filed on Dec. 16, 2002.

(51) Int. Cl. A61B 17/58

(52) U.S. Cl. 606/73

(57) ABSTRACT

A dental or medical implant (10, 20, 30) has a cortical bone threaded axial length portion (10b) located between an unthreaded axial length apical portion (10c) and an unthreaded axial length coronal portion (10d). The implant is formed with an integrally formed abutment head (10g, 20a, 30a) with selected head configurations.

### CROSS REFERENCE TO RELATED APPLICATIONS

Benefit is claimed, under 35 U.S.C. section 111(b), of Provisional Application No. 60/433,659 filed Dec. 16, 2002.

### BACKGROUND OF THE INVENTION

Implants that can be screwed into bone are well known in the art. For example, one such screw implant is shown in U.S. Pat. No. 6,325,628. In that particular patent a combination implant and abutment member is shown particularly useful for mounting temporary prosthesis comprising a head portion integrally formed on an implant post having cortical bone threads formed along the length of the post.

An object of the present invention is the provision of a threaded implant having improved biocompatibility of the implant and attached prosthetics.

### SUMMARY OF THE INVENTION

Briefly, an implant made in accordance with the invention comprises an elongated post member having an integrally formed head at one end and a threaded axial length portion disposed between unthreaded apical and coronal axial length portions. The coronal axial length portion preferably includes a neck formed with an epithelial stop and a taper that serves to seal the osteotomy into which the implant member is placed. Several head configurations are shown which include a smooth convexly curved basal portion on which an angled shelf is formed. One head configuration has a snap-on O-ring groove and hexagonal feature formed on a nose portion projecting from the shelf. Another head configuration has a coping receiving conical surface formed on a nose portion projecting from the shelf and a threaded bore formed through the outer free end surface of the nose for mounting a temporary prosthesis. Still another head configuration has a generally conically shaped nose projecting from the shelf and formed with first and second axial length portions. A stepped retention feature for a sleeve member or the like is provided by forming a larger circumference of the apical side of the outer axial length portion compared to the circumference of the coronal side of the inner axial length portion with both portions having a diameter that decreases in the direction along the axial lengths going away from the shelf.

Additional objects and features of the invention will be set forth in part in the description which follows and in part will be obvious from the description. The objects and advantages of the invention may be realized and attained by means of the instrumentalities and methods particularly pointed out in the appended claims.

### BRIEF DESCRIPTION OF THE DRAWINGS

The accompanying drawings, which are incorporated in and constitute a part of the specification, illustrate preferred embodiments of the invention and, together with the description, serve to explain the objects, advantages and principles of the invention. In the drawings:

FIG. 1 is a front view of an implant member made in accordance with a first preferred embodiment of the invention and FIG. 1a is a top view thereof;

FIG. 2 is a front view of an implant member made in accordance with a second preferred embodiment and FIG. 2a is a top view thereof; and

FIG. 3 is a front view of an implant member made in accordance with a third preferred embodiment and FIG. 3a is a top view thereof.

### DETAILED DESCRIPTION OF THE PREFERRED EMBODIMENTS

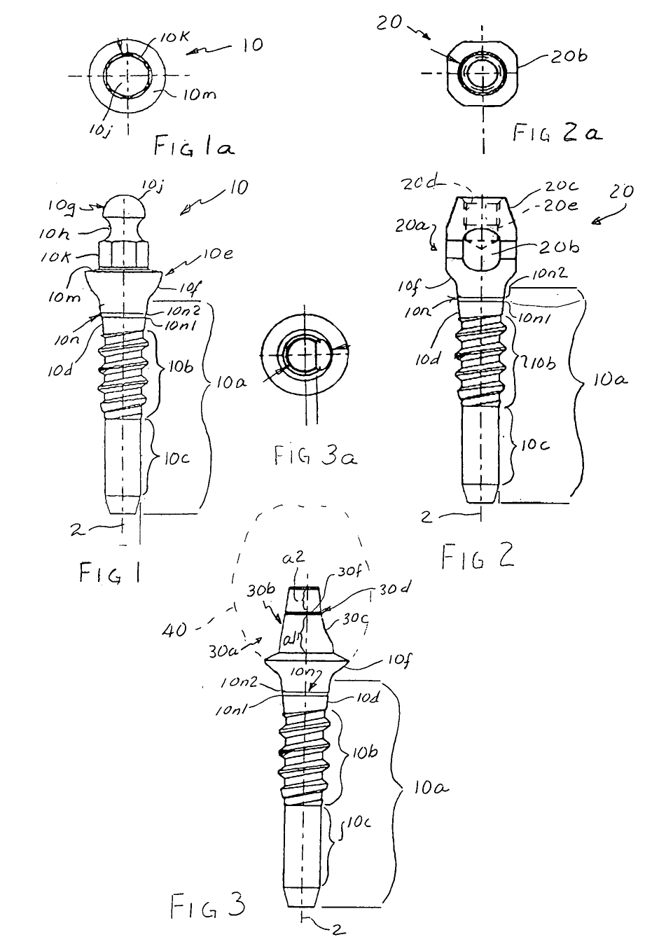

With reference to FIGS. 1 and 1a, implant member 10 comprises an elongated post having a selected axial length 10a, two such examples being 0.315 inch and 0.472 inch, and having a selected diameter such as 0.073 inch, formed with a cortical bone screw thread along a first significant or major axial length portion 10b intermediate to an unthreaded apical second significant or major axial length portion 10c and an unthreaded coronal third axial length portion 10d. Unthreaded apical axial length portion 10c can be of any selected length, for example a length generally corresponding to the axial length portion 10b of the cortical bone thread, as shown in the drawings. Implant member 10 is also formed with an integral abutment portion 10e. Abutment portion 10e has a basal portion 10f formed with a smooth, convex, outer surface configuration, such as spherical, elliptical or the like, and a head portion 10g. Head portion 10g is formed with a nose portion projecting from a shelf 10m. The nose portion has a circumferentially extending O-ring groove 10h between a generally semispherical outer free end 10j and a polygonal, such as hexagonal, configuration 10k adjacent to the shelf. Shelf 10m extends from the junction of head 10j with basal portion 10f. Shelf 10m preferably forms an angle with an imaginary plane perpendicular to the longitudinal axis 2 of the head portion in the range of approximately 0-30 degrees, and more preferably, approximately 15 degrees, as disclosed and discussed in U.S. Pat. No. 6,290,500, the subject matter of which is incorporated herein by this reference.

Head portion 10g is particularly useful for mounting prosthesis having bridge structures and the like formed with O-ring snap-on fixation attachment structures. Polygonal configuration portion 10k can be used for driving the member into an osteotomy in a known manner.

Preferably, an epithelial stop 10n is provided comprising an annular surface circumscribing unthreaded coronal portion 10d of implant member 10. Epithelial stop 10n comprises an annular surface groove that forms a sharp angle with the outer peripheral surface, e.g., a right or acute angle on the apical side of the groove. Annular groove surface portion 10n2 extends from the outer peripheral surface of coronal portion 10d on the coronal side to the inner part of the groove in a direction toward the apical end of the implant member and forms a relatively less sharp, i.e., a relatively smooth, transition with the outer peripheral surface of coronal portion 10d. Annular surface portion 10n1 forms a second surface portion which extends from the inner part of the groove generally toward the coronal end of the implant member to form a sharp transition with the outer peripheral surface of the coronal portion 10d. Such epithelial stops are disclosed in U.S. Pat. No. 6,227,857, the subject matter of which is incorporated herein by this reference.

Coronal portion 10d is preferably formed with a taper with the diameter of that portion increasing as one proceeds in a direction toward the basal portion of the implant member which serves to seal the osteotomy into which the implant member is placed. The unthreaded coronal portion also allows for adaptating to varying mucosal thicknesses.

The above described structure of the cortical bone threaded portion disposed between the unthreaded apical and coronal portions results in the following advantages:

The unthreaded apical portion serves as a pilot portion and provides a structure which requires fewer turns to seat the implant member into a prepared pilot bore or hole in a bone.

The unthreaded apical portion serves to maintain the implant in a path following the trajectory of the pilot bore.

The unthreaded apical portion provides lateral stability without applying pressure on the bone that could result in initial osteoclastic bone activity.

The unthreaded apical portion allows for immediate bone deposition or growth without undergoing osteoclastic bone activity.

As noted above, the unthreaded coronal portion allows for adapting to varying mucosal thicknesses.

As noted above, the tapered neck of the coronal portion of the post serves to seal the osteotomy into which the implant member is placed.

The implant member can be provided with various head structures, for example, as shown in FIGS. 2, 2a and 3, 3a, to be described below.

FIGS. 2 and 2a show an implant member 20 made in accordance with a second preferred embodiment of the invention. Implant member 20 is formed with a head 20a having a cylindrical nose projecting from basal portion 10f and has four spaced apart flats 20b to facilitate driving the member into an osteotomy formed in the cortical bone. It will be understood that the number of flats provided is a matter of choice. A tapered or conical coping receiving surface 20c is formed at the coronal end portion and a bore 20d is formed through the coronal end surface extending along the longitudinal axis 2. The outer portion of bore 20d is threaded and the inner portion 20e may be formed with a non-circular, e.g., hexagonal, portion to facilitate fastening in a bore by means of a latched dental hand piece screw driver. A coping (not shown), typically formed of suitable moldable plastic material to which a temporary prosthesis (not shown) will bond, can be placed on head 20a of several such implant members for supporting a temporary or permanent prosthesis, being attached by means of threaded pins received in bore 20d of the implant members. A system of this type is disclosed in U.S. Pat. No. 6,325,628, referenced above, the subject matter of which is incorporated herein by this reference. The basal portion 10f and axial length post portion 10a are the same as in the FIGS. 1, 1a embodiment to which reference may be had for a description thereof.

If desired, head 20a can be formed with bores (not shown) through the head to accommodate the fastening of orthopedic wires. For example, two bores extending through the head generally perpendicular to the longitudinal axis 2 at a selected angle to each other, e.g., 90 degrees, and preferably at different axial levels to thereby provide anchorage for orthodontic wires and devices.

FIGS. 3 and 3a show an implant member 30 for mounting crowns and the like made in accordance with a third preferred embodiment of the invention. Implant member 30 is formed with a head 30a having a generally cylindrical, tapered nose 30b projecting from shelf 10m. Nose 30b is formed with one or more anti-rotation flats 30c and preferably with a stepped locking or retention portion 30d formed intermediate to first and second axial length portions a1, a2 of nose 30b and preferably separated from each other by a circumferentially extending groove 30f. Stepped axial length portions can be formed to extend completely around the circumference, or, if desired, along a portion of thereof, such as along the radiused portion of nose portion 30b, e.g., portions of the circumference between the one or more anti-rotation flats. The circumference of each axial length portion decreases as the distance from the basal portion 10f increases. Further, the circumference of nose portion 30b on the coronal side of the stepped portion, or the side further from the basal portion, immediately adjacent to groove 30f, is larger than the circumference of the nose on the apical side of the groove to form the retention feature. By way of example, an implant member made in accordance with the FIGS. 3, 3a embodiment has an apical diameter of axial length a2 of 0.076 and the coronal diameter of axial length a1 of 0.073. Abutment heads of this type are disclosed in U.S. Pat. No. 6,592,370, the subject matter of which is incorporated herein by this reference. The basal portion 10f and implant post portion 10a are the same as in the FIGS. 1 and 1a embodiment to which reference may be had for a description thereof.

Also shown in FIG. 3 is a typical crown 40 shown in dashed lines that can be attached to implant member 30, as by cementing thereto in a known manner.

It will be understood that still other head configurations can be used with implant post 10a within the purview of the invention.

Although the invention has been described with regard to specific preferred embodiment thereof, variations and modifications will become apparent to those skilled in the art. It is therefore, the intention that the appended claims be interpreted as broadly as possible in view of the prior art to include all such variations and modifications.

What is claimed:

1. An implant member for threaded reception in an osteotomy in a bone comprising an elongated generally cylindrical post having a longitudinal axis, a coronal end and an apical end, the coronal end having an abutment portion having a smoothly curved basal portion integrally formed at the coronal end of the post, cortical bone threads formed along a first significant axial length portion of the post proximate to the abutment portion and an unthreaded, second significant axial length apical portion contiguous to the first axial length portion.

2. An implant member according to claim 1 further comprising a third smooth, non-threaded axial length coronal portion between the basal portion and the threaded first significant axial length portion.

3. An implant member according to claim 2 in which the third axial length portion is formed with a slight sealing taper of increasing diameter moving in the direction from the apical end toward the coronal end.

4. An implant member according to claim 3 further comprising an epithelial stop in the form of a circumferential groove formed in the tapered portion of the third axial length portion, the groove forming a sharply angled transition with the outer peripheral surface of the third axial length portion on the apical side of the groove and a relatively smoothly angled transition with the outer peripheral surface of the third axial length portion on the coronal side of the groove.

5. An implant member according to claim 1 in which the first and second axial length portions are generally equal in length.

6. An implant member according to claim 1 in which the abutment portion includes a nose projecting from the basal portion, the nose being formed with first and second axial length portions each of which is tapered with a decreasing diameter moving in a direction from the apical end to the coronal end of the post, a retention step being formed between the first and second axial length portions of the nose, the first axial length portion being contiguous with the basal portion and having a circumference on the coronal side of the retention step less than the circumference of the second axial length portion on the apical side of the retention step.

7. An implant member according to claim 1 in which the abutment portion includes a nose projecting from the basal portion, the nose having a generally spherical free end and an O-ring groove formed adjacent to the spherical free end.

8. An implant member according to claim 1 in which the abutment portion includes a nose projecting from the basal portion and the nose has a free end formed with a tapered coping receiving surface circumscribing the free end and a threaded bore is formed along the longitudinal axis through the free end of the nose.
